(19) United States
(12) Patent Application Publication
(10) Pub. No.: US 2004/0106548 A1
(43) Pub. Date: Jun. 3, 2004

(54) Conformationally constrained labeled peptides for imaging and therapy

(76) Inventors: Michelle A Schmidt, Belleville, IL (US); Jack L Erion, St Charles, MO (US); Ananthachari Srinivasan, Bedford, NH (US)

Correspondence Address: BLACKWELL SANDERS PEPER MARTIN LLP, 720 OLIVE STREET, SUITE 2400, ST. LOUIS, MO, 63101, US

(21) Appl. No.: 10/363,977

(22) PCT Filed: Sep. 7, 2001
(86) PCT No.: PCT/US01/27708

(51) Int. Cl. A61K 38/23

(52) U.S. Cl. 514/12

(57) ABSTRACT

Conformational constraints in diagnostic and therapeutic agents in peptides have been introduced by utilization of disulfide bonds and amide cyclizations. These constraints are responsible for altering the stability and specificity of these receptor-targeted agents. Conformationally constrained peptides containing secondary and primary amines, ethers, thioethers, amidines, esters and other functionalities have been synthesized. Methods are disclosed which incorporate multiple features of the above functionalities in the macrocyclic ring of the peptides.

### FIELD OF INVENTION

The present invention relates to conformationally constrained receptor targeted radiolabeled peptides which are amenable to positioning of a chelating moiety (henceforth referred to as “CM”) and/or diagnostic or therapeutic isotopes. The methodology is applicable to several families of peptides including but not limited to: somatostatin, gastrin, gastrin releasing peptide, bombesin and bombesin antagonists, gastrin releasing peptides, adhesion peptides, cholecystokinin, neurotensins, neuropeptide Y, vasoactive intestinal peptides, thyroid stimulating hormone, angiotensin, pancreatic adenylate cyclase activating peptide, and substance P. Instead of chelating moieties containing diagnostic and therapeutic isotopes, other diagnostic agents such as fluorescent dyes, dyes that absorb at the near infrared region, can be attached at the same position. Also, instead of chelating moieties, other therapeutic agents such as physiologically acceptable drugs can be attached at the same position.

### BACKGROUND OF THE INVENTION

Over the years, the presence of various receptors has been demonstrated in a wide variety of tumors. Diagnostic agents based on peptides have been introduced: In-111-DTPA-somatostatin analogs (see U.S. Pat. Nos. 5,753,627 and 5,776,894) were introduced for the purpose of imaging and therapy of somatostatin subtype-2 receptors. In this case, all the chelating moieties were attached to the N-terminus of the peptides. The proteins or antibodies were either radioiodinated or reacted with bifunctional chelating agents and randomly substituted.

### SUMMARY OF THE INVENTION

Conformational constraints in diagnostic and therapeutic agents in peptides have been introduced by means of disulfide bonds and amide cyclizations. These constraints are responsible for altering the stability and specificity of these receptor-targeted agents.

Conformationally constrained peptides containing secondary and primary amines, ethers, thioethers, amidines, esters and other functionalities have been synthesized. Methods are disclosed which provide means for incorporating multiple features of the above functionalities in the macrocyclic ring of the peptides.

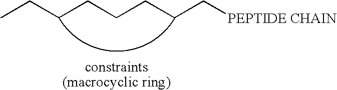

In many instances, there is a specific need for attachment of the chelating moieties away from the binding sites (besides the N-terminus, C-terminus and side chains of the amino acid sequences). Incorporation of amines in the macrocyclic ring provides a handle for the incorporation of the chelating moiety away from the binding sites. Incorporation of ether and thioether and other functionalities allows isosteric substitution of the macrocyclic ring. Incorporation of esters in the macrocyclic ring provides stability to the ring and a means towards rapid degradation and elimination after localization in the excretionary organs. It is understood that a combination of the above features can be incorporated between any two positions of the amino acid chain of the peptide. In addition to or in place of attaching chelating moieties, other moieties may be attached to the macrocyclic ring. Such other moieties include, but are not limited to, dyes which are useful for detection such as for diagnostic purposes and drugs which can be used for therapeutic purposes.

### DETAILED DESCRIPTION OF THE INVENTION

It is well known in the field of peptide chemistry that cyclization of peptides alters stability and specificity of the peptides. The conformation of a peptide can be stabilized or fixed by the introduction of a ring. In several naturally occurring peptides, the conformation is stabilized by the presence of disulfide or lactam bridges. Peptides containing disulfide bridges undergo metabolism with the formation of cysteines followed by enzymatic degradation of the peptide. Isosteric substitution of the disulfide bridge with either CH₂—S or CH₂—CH₂ bridge should not only inhibit metabolism of the peptide, but also prolong the serum half-life of the peptide. Such a modification, however, may also render rigidity to the ring resulting in an inactive compound.

Incorporation of varying ring sizes (macrocyclic chain) between the side chains of amino acids renders different three-dimensional conformations of the peptide chain. These features can impose different specificities for the peptide. The incorporation of O, S or NH alters flexibility of the macrocyclic chain, while amines (endocyclic and exocyclic) can also be utilized for incorporation of diagnostic and therapeutic entities (radiolabeled chelating groups, dyes and chemotherapeutic drugs). Incorporation of esters (lactones) allows temporary serum-stability and imparts specificity to the peptide, while aiding metabolism in the exeretionary organs.

We have already demonstrated that the DTPA-monosulfide analog with an identical ring size maintains tumor targeting. Hence, a new chemical method was sought to prepare carbocyclic peptides preferably in the solid phase. Such a method should also be amenable for combinatorial chemistry to prepare a wide variety of cyclic peptides with or without functional groups in the ring as well as peptidomimetics.

Since its introduction ten years ago, the ring closing metathesis reactions catalyzed by Ru, Mo and Ti carbene complexes have been used to synthesize a wide variety of carbocyclic and heterocyclic compounds. In simple terms, olefin metathesis is a carbon skeleton redistribution in which new unsaturated carbon-carbon bonds are formed in the presence of metal catalysts.

The ring closing metathesis of a diene involves an alternating type of propagation reaction. An intermolecular metathesis reaction with the carbene complex is followed by an intramolecular metathesis reaction. The ease of occurrence of both these steps varies, and the stereoselectivity of the cyclization step varies with the catalyst. The optimum condition for a given ring closing metathesis must be found by trial and error.

The substrate concentration plays a major role in the success of any ring closing metathesis reaction. Dilute solution, favors the intramolecular reaction. Since a protected peptide attached to a solid phase can be considered a pseudo-dilute solution, ring closing metathesis typically favors intramolecular cyclization. The success of the reaction depends on several factors. If there are chiral centers between two reacting multiple bonds, then the ring closing metathesis of one diastereomer may be favored over the other.

Abbreviations used in this disclosure are as follow: Dab is diaminobutyric acid; AGly is α-allylglycine; All is allyl; Dde is 1-(4,4-dimethyl-2,6-dioxocyclohexylidine)-ethyl; Ph₃P is triphenylphosphine; DEAD is diethylazodicarboxylate; NBS is o-nitrobenzenesulfonyl; HOBt is N-hydroxybenzotriazole; HBTU is 2-(1H-benzotriazole-1-yl)-1,1,3,3-tetramethyluronium hexafluorophosphate; DBU is diazabicycloundecane; MeOtBu is methyl-t-butyl ether and TFA is trifluoroacetic acid. Common amino acids are given their common three letter codes. Unless otherwise stated, the amino acids have the L-configuration at the chiral center.

Resin-bound, protected α-allylGly^{2,7}-Octreotate (AGly^{2,7}-Octreotate) was cyclized to the unsaturated compound in the presence of Grubbs' catalyst (bis(triscyclohexylphosphine)benzylidine ruthenium (IV) dichloride). After the introduction of the chelating moiety, deprotection and reduction yielded the carbocyclic peptide. However, isosteric replacement of the disulfide with a carbocyclic bridge imparts rigidity to the peptide. This results in the loss of binding affinity to the somatostatin receptors. Presence of a carbocyclic bridge presents the binding region of the peptide in an unfavorable conformation to the receptor. Hence, methods to enlarge the ring to relieve the rigidity were sought. Resin-bound, protected AGly²,Ser(OAll)⁷-Octreotate and Ser(OAll)^{2,7}-Octreotate were cyclized to the unsaturated compound in the presence of Grubbs' catalyst. After the introduction of the chelating moiety, deprotection and reduction yielded the macrocyclic peptide.

Metathesis reaction of protected, resin-bound Fmoc-AGly³,Glu(γ-OAll)⁷-Octreotate gave >90% of the intramolecular cyclic ester when the resin loading was 0.18 mmol/g. This resin-bound peptide was deprotected, and DTPA was incorporated using tri-t-butyl-DTPA anhydride. When the resin loading was 0.5 mmol/g, an intermolecular metathesis reaction occurred. Based on the molecular weight, the compound was assigned the dimeric structure. This is the first observation of an intermolecular metathesis reaction in solid phase, and the course of the reaction can be altered depending on the resin loading. This observation was used to prepare dimers of somatostatin and adhesion (αᵥβ₃) peptides. This method is generally applicable to other peptides mentioned earlier (gastrin, gastrin releasing peptide, bombesin and bombesin antagonists, gastrin releasing peptides, cholecystokinin, neurotensins, neuropeptide Y, vasoactive intestinal peptides, thyroid stimulating hormone, angiotensin, pancreatic adenylate cyclase activating peptide, other adhesion peptides and substance P). Catalytic reduction of the cyclic, unsaturated ester led to ring opening to yield DTPA-Gly(α-Bu)³,Glu⁷-Octreotate.

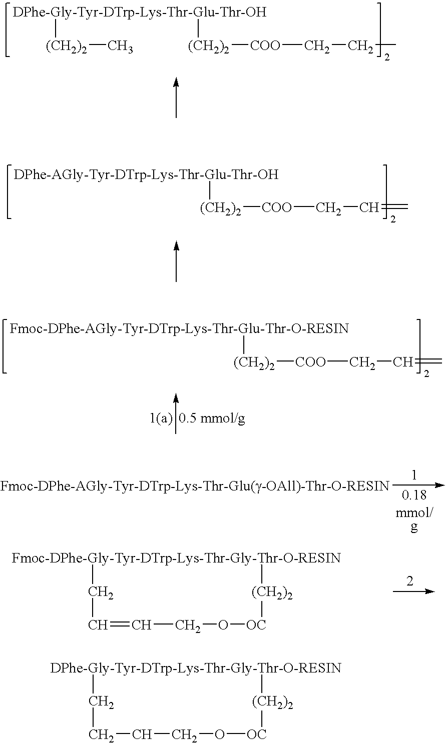

Metathesis reaction of linear bombesin and neurotensin derivatives resulted in cyclic esters.

We have developed a method of stabilizing arginines by using amidine nitrogen to provide stabilization and to provide specificity. This method can be used for any arginine containing peptide, including those with an Arg-Gly-Asp (RGD) sequence. Metathesis reactions can be performed on RGD containing peptides. RGD containing peptides have been implicated as inhibitors of integrin-ligand interaction in studies of cell adhesion, migration and differentiation. In the present literature, all of the Arg-Gly-Asp peptides are either linear or the sequence is contained within a cyclic structure to provide stability and specificity. The methods disclosed herein use the Arg amidine nitrogen to stabilize the conformation of the RGD molecules. This arrangement results in stability against enzymatic degradation.

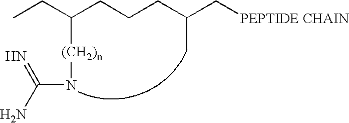

The discussion above, together with the specific examples discussed below, show that a metal complex-catalyzed metathesis reaction has been successfully utilized for the synthesis of carbocyclic, cyclic ethers and cyclic esters. An intermolecular metathesis reaction in solid phase was observed in some instances at high loading levels of the resin, but intramolecular cyclizations are favored at lower loading levels. The methods developed for somatostatin peptides are applicable to other peptides as exemplified by the preparation of neurotensin and bombesin peptides. The reaction conditions are designed to prepare a wide variety of cyclic compounds for functionalization either at the N-terminus or in the macrocyclic ring. Macrocyclic peptides are ideal candidates for Tc-99m chelation chemistry because of the absence of reducible groups, such as disulfide. Methods developed here are amenable to the preparation of a large number of peptides and peptidomimetics by combinatorial chemistry.

I. Endocyclic Amines Containing a Chelating Moiety

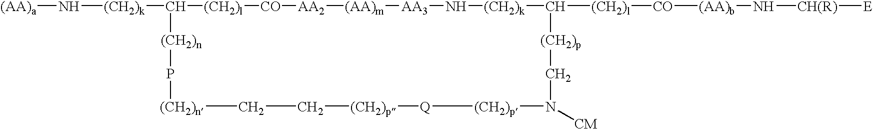

AA, AA₂, AA₃=natural and unnatural amino acids; this includes α-, β- and Γ-aminoacids and L- and D-aminoacids;

a,b=0-10;

k,l=0-5;

m=0-20;

n,n′=1-10;

P is none, O, S, COO, NH—CO, NR, N—CH(═NH)—NH₂, NH—CO—NH, NH—COO;

R is hydrogen or C₁-C₅ linear or branched chain alkyl groups bearing —OH at any location;

p, p′, p″=0-10;

Q is none, O, S, COO, NH—CO, NR, N—CH(═NH)—NH₂, NH—CO—NH, NH—COO;

E is a group of formula COOR₄, CH₂OR₅, CON(R₆)OH or CON(R₇)(R₈) wherein

R₄ is hydrogen or C₁-C₅ linear or branched chain alkyl groups,

R₅ is hydrogen or physiologically acceptable, physiologically hydrolyzable ester,

R₆ is hydrogen or C₁-C₅ linear or branched chain alkyl groups,

R₇, R₈ is hydrogen or C₁-C₅ linear or branched chain alkyl groups or taken together form a cyclic alkyl group C₃-C₁₀;

CM is a dye, a therapeutic agent, or a chelating moiety or metal binding site wherein the chelating moiety is labeled with a metal isotope selected from ^{99m}Tc, ²⁰³Pb, ⁶⁷Ga, ¹¹¹In, ⁹⁷Ru, ⁶²Cu, ⁶⁴Cu, ¹⁸⁶Re, ¹⁸⁸Re, ⁹⁰y, ¹²¹Sn, ¹⁶¹Tb, ¹⁵³Sm, ¹⁶⁶Ho, ¹⁰⁵Rh, ¹⁷⁷Lu or a radioactive halogen isotope on the understanding that

i) if the label is a metal isotope, CM represents a chelating group suitable for the metal and

ii) if the label is a radioactive halogen isotope, the halogen is attached to an aromatic ring,

wherein the CM is attached directly or through a spacing group to the peptide, said CM being attached to the amine through an amide or urea bond or by any other modification which allows attachment of a chelate and which modifications are known to those of skill in the art,

wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA) or a compound with a general formula

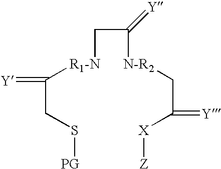

wherein

PG is a sulfur protecting group selected from alkanoyl, arylcarbonyl, arylalkanoyl, acetamidomethyl, tetrahydropyranyl and tetrahydrofuranyl,

Y′, Y″, and Y′″ are hydrogen or oxygen with the proviso that at least one of them is an O,

R₁ and R₂ are hydrogen or alkyl (C₁-C₃),

X═NH or S with the proviso that Y′″ is hydrogen when X is S,

Z is PG if X is S, and

Z is hydroxyalkyl, aminoalkyl or carboxyalkyl.

II. Exocyclic Amines Containing a Chelating Moiety

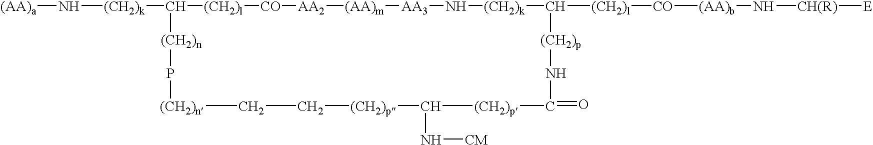

AA, AA₂, AA₃=natural and unnatural amino acids; this includes α-, β- and γ-aminoacids and L- and D-aminoacids;

a,b=0-10;

k,l=0-5;

m=0-20;

n, n′=1-10;

P none, O, S, COO, NH—CO, NR, N—CH(═NH)—NH₂, NH—CO—NH, NH—COO;

R is hydrogen or C₁-C₅ linear or branched chain alkyl groups bearing —OH at any location;

p, p′, p″=0-10;

E is a group of formula COOR₄, CH₂OR₅, CON(U)OH or CON(R₇)(R₈) wherein

R₄ is hydrogen or C₁-C₅ linear or branched chain alkyl groups,

R₅ is hydrogen or physiologically acceptable, physiologically hydrolyzable ester,

R₆ is hydrogen or C₁-C₅ linear or branched chain alkyl groups,

R₇, R₈ is hydrogen or C₁-C₅ linear or branched chain alkyl groups or taken together form a cyclic alkyl group C₃-C₁₀;

CM is a dye, a therapeutic agent, or a chelating moiety or metal binding site wherein the chelating moiety is labeled with a metal isotope selected from ^{99m}Tc, ²⁰³Pb, ⁶⁷Ga, ¹¹¹In, ⁹⁷Ru, ⁶²Cu, ⁶⁴Cu, ¹⁸⁶Re, ¹⁸⁸Re, ⁹⁰Y, ¹²¹Sn, ¹⁶¹Tb, ¹⁵³Sm, ¹⁶⁶Ho, ¹⁰⁵Rh, ¹⁷⁷Lu or a radioactive halogen isotope on the understanding that

i) if the label is a metal isotope, CM represents a chelating group suitable for the metal and

ii) if the label is a radioactive halogen isotope, the halogen is attached to an aromatic ring,

wherein the CM is attached directly or through a spacing group to the peptide, said CM being attached to the amine through an amide or urea bond or by any other modification which allows attachment of a chelate and which modifications are known to those of skill in the art,

wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA) or a compound with a general formula

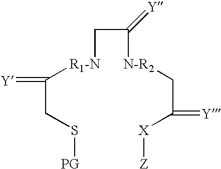

wherein

PG is a sulfur protecting group selected from alkanoyl, arylcarbonyl, arylalkanoyl, acetamidomethyl, tetrahydropyranyl and tetrahydrofuranyl,

Y′, Y″, and Y′″ are hydrogen or oxygen with the proviso that at least one of them is an O,

R₁ and R₂ are hydrogen or alkyl (C₁-C₃),

X═NH or S with the proviso that Y′″ is hydrogen when X is S,

Z is PG if X is S, and

Z is hydroxyalkyl, aminoalkyl or carboxyalkyl.

III. Chelating Moiety at the N-Terminus

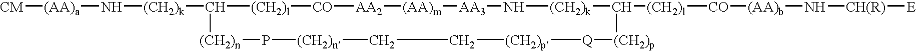

AA, AA₂, AA₃=natural and unnatural amino acids; this includes α-, β- and γ-aminoacids and L- and D-aminoacids;

a,b=0-10;

k,l=0-5;

m=0-20;

n, n′=1-10;

P, Q is none, O, S, COO, NH—CO, NR, N—CH(═NH)—NH₂, NH—CO—NH, NH—COO;

R is hydrogen or C₁-C₅ linear or branched chain alkyl groups bearing —OH at any location;

p, p′=0-10;

E is a group of formula COOR₄, CH₂OR₅, CON(R₆)OH or CON(R₇)(R₈) wherein

R₄ is hydrogen or C₁-C₅ linear or branched chain alkyl groups,

R₅ is hydrogen or physiologically acceptable, physiologically hydrolyzable ester,

R₆ is hydrogen or C₁-C₅ linear or branched chain alkyl groups,

R₇, R₈ is hydrogen or C₁-C₅ linear or branched chain alkyl groups or taken together form a cyclic alkyl group C₃-C₁₀;

CM is a dye, a therapeutic agent, or a chelating moiety or metal binding site wherein the chelating moiety is labeled with a metal isotope selected from ^{99m}Tc, ²⁰³Pb, ⁶⁷Ga, ¹¹¹In, ⁹⁷Ru, ⁶²Cu, ⁶⁴Cu, ¹⁸⁶Re, ¹⁸⁸Re, ⁹⁰Y, ¹²¹Sn, ¹⁶¹Tb, ¹⁵³Sm, ¹⁶⁶Ho, ¹⁰⁵Rh, ¹⁷⁷Lu or a radioactive halogen isotope on the understanding that

i) if the label is a metal isotope, CM represents a chelating group suitable for the metal and

ii) if the label is a radioactive halogen isotope, the halogen is attached to an aromatic ring,

wherein the CM is attached directly or through a spacing group to the peptide, said CM being attached to the amine through an amide or urea bond or by any other modification which allows attachment of a chelate and which modifications are known to those of skill in the art,

wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA) or a compound with a general formula

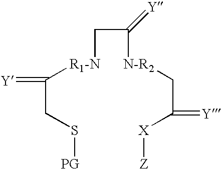

wherein

PG is a sulfur protecting group selected from alkanoyl, arylcarbonyl, arylalkanoyl, acetamidomethyl, tetrahydropyranyl and tetrahydrofuranyl,

Y′, Y″, and Y′″ are hydrogen or oxygen with the proviso that at least one of them is an O,

R₁ and R₂ are hydrogen or alkyl (C₁-C₃),

X═NH or S with the proviso that Y′″ is hydrogen when X is S,

Z is PG if X i s S,and

Z is hydroxyalkyl, aminoalkyl or carboxyalkyl.

Throughout this disclosure, the dyes and therapeutics which can be used for CM include, but are not limited to, the following:

Visible dyes:

Fluorescein

Fluorescein isothiocyanate (FITC)

Naphthofluorescein

Rhodamine derivatives

Texas Red

Hydroxycoumarin

Infrared dyes:

Indocyanine Green (ICG)

Bis-propanoic acid cyanine

Photodynamic therapy dyes/photosensitizers:

Acridines (acridine orange, acridine yellow, proflavin, etc.)

Thiazines (methylene blue, azure C, toluidine blue)

Xanthenes (fluorescein, rose Bengal)

Phenazines (neutral red)

Porphyrins

Naphthalimide

Cancer Drugs:

Tamoxifen

Adriamycin

Phillotoxins

Taxol and analogs

Bleomycin

Doxorubicin

Etoposide

Methotrexate

Vinblastine and analogs

Dicarbazine

Actinomycin D

The invention will now be described in greater detail with reference to the following specific Examples, which are offered by way of illustration and are not intended to limit the invention in any manner. Standard techniques well known in the art or the techniques specifically described below are utilized.

### EXAMPLE 1

Peptide Synthesis

All the linear peptides in the study were prepared by solid phase peptide synthesis employing a Fmoc[9-fluorenylmethoxycarbonyl] strategy. All the amino acids were purchased commercially.

In all the following examples, in all the resin bound peptides, the side chains of the individual amino acids have protecting groups unless otherwise stated.

### EXAMPLE 2

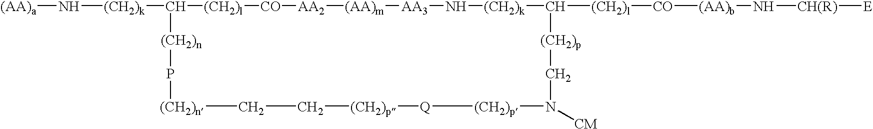

Somatostatins:

(AA)ₐ is Phe, Tyr, an isomer of Tyr, polyhydroxylated Phe, or aromatic amino acids, wherein the amino acid can have an L- or D-configuration;

k is 1, 2or 3;

l is 1, 2or 3;

AA₂ is Phe, Tyr, an isomer of Tyr, polyhydroxylated Phe, or aromatic amino acids, wherein the amino acid can have an L- or D-configuration;

(AA)ₘ is a dipeptide sequence consisting of DTrp-Lys, DTrp-Orn, DTrp-Dab, DTrp-4-piperidinylglycine, DTrp-4-piperidinylalanine, DTrp-4-aminomethylcyclohexylalaninie, DTrp-4-aminomethylcyclohexylglycine, DTrp-4-aminocyclohexylalanine, DTrp-4-aminocyclohexylglycine. DTrp can be substituted by L-Trp;

AA₃ is any amino acid;

(AA)_{b} is none, serine or threonine;

R is hydrogen or C₁-C₅ linear or branched chain alkyl groups bearing —OH at any location;

E is COOH, CH₂—OH, CONH₂, COOR₄ or CONHOH wherein R₄ is hydrogen or C₁-C₅ linear or branched chain alkyl groups;

n is 1, 2 or 3;

P is none, O or S;

n′ is 1-7;

p is 1-6;

p′ is 1-6;

p″ is 1-6;

Q is none, O or S;

CM is a dye, a therapeutic agent, or a chelating moiety or metal binding site wherein the chelating moiety is labeled with a metal isotope selected from ^{99m}Tc, ²⁰³Pb, ⁶⁷Ga, ¹¹¹In, ⁹⁷RU, ⁶²Cu, ⁶⁴Cu, ¹⁸⁶Re, ¹⁸⁸Re, ⁹⁰Y, ¹²¹Sn, ¹⁶¹Tb, ¹⁵³Sm, ¹⁶⁶Ho, ¹⁰⁵Rh, ¹⁷⁷Lu or a radioactive halogen isotope on the understanding that

i) if the label is a metal isotope, CM represents a chelating group suitable for the metal and

ii) if the label is a radioactive halogen isotope, the halogen is attached to an aromatic ring,

wherein the CM is attached directly or through a spacing group to the peptide, said CM being attached to the amine through an amide or urea bond or by any other modification which allows attachment of a chelate and which modifications are known to those of skill in the art,

wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA) or a compound with a general formula

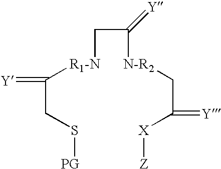

wherein

PG is a sulfur protecting group selected from alkanoyl, arylcarbonyl, arylalkanoyl, acetamidomethyl, tetrahydropyranyl and tetrahydrofuranyl,

Y′, Y″, and Y′″ are hydrogen or oxygen with the proviso that at least one of them is an O,

R₁ and R₂ are hydrogen or alkyl (C₁-C₃),

X═NH or S with the proviso that Y′″ is hydrogen when X is S,

Z is PG if X is S, and

Z is hydroxyalkyl, aminoalkyl or carboxyalkyl.

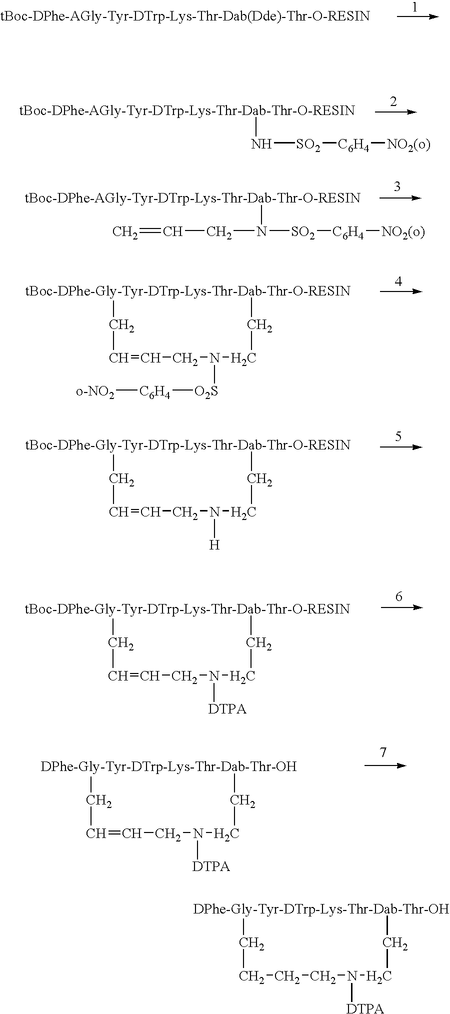

Step 1: The protected peptide was assembled in an automated synthesizer according to the Fmoc-strategy. The resin (Wang) bound peptide was shaken with 2% hydrazine (2 mL hydrazine per 50 mg of resin) for 30 minutes to remove the Dde protecting group, followed by protection of the side chain amino group with o-nitrobenzenesulfonyl group (NBS) using commercially available o-nitrobenzenesulfonyl chloride in the presence diisopropylethylamine (DIEA).

Step 2: The resin, tBoc-DPhe¹,AGly²,Tyr³,Dab⁷(β-o-NBS)-Octreotate-Resin (55 mg, 10 μmol peptide content, 0.18 mmol/g) was suspended in a solution of 1 mL of methylene chloride CH₂Cl₂ containing 52 mg of triphenyl phosphine (Ph₃P) (0.2 mmol; 20 Xs.) 34 μL of diethylazodicarboxylate (DEAD) (0.2 mmol; 20 Xs.). After vigorous shaking for a few minutes, allyl alcohol (20 fold excess) was added. After vortexing for overnight, the resin was filtered, washed with 5 mL of methylene chloride and dried. In similar reactions, the resin-bound peptide was alkylated with 3-butenol, 4-pentenol, 5-hexenol or allyloxyethanol. In each case, a small amount of the peptide was cleaved from the resin and assayed to ensure complete alkylation.

Step3: 50 mg of the resin (25 μmole peptide) was suspended in 5 mL of methylene chloride containing 20 mg of Grubbs' catalyst. The mixture was heated at 40° C. for 10-15 hours. At the end of the reaction, the resin was removed by filtration and washed with methylene chloride and THF (tetrahydrofuran).

Step 4: The resin (250 mg; 0.18 mmol/g) containing the previously made peptide of step 3 was suspended in 3 mL of DMF (dimethylformamide). To this suspension, 200 μL of DBU and 200 μL of mercaptoethanol was added and shaken for 7 hours.

Step 5: A solution of tri-t-butyl-DTPA anhydride (56 mg; 0.1 mmol) in 200 μL of DMF was activated with 0.5 mL of HOBt-HBTU (200 mM) solution for 1 hour and added to 140 mg (50 μmol of the peptide) of the above resin. The suspension was shaken for overnight and filtered. The resin was washed with DMF and 10 mL of THF.

Step 6: The resin was deprotected using 250 μL of TFA:phenol:thioanisole:water (85:5:5:5) overnight. The crude peptide was precipitated using 10 mL of MeOtBu. After centrifugation, the mixture was washed with 4×10 mL of dissolved in MeOtBu. The mixture was taken up in 2 mL of 2:3 acetonitrile:water, shaken in a vortex mixer and the resin was removed by filtration. The filtrate was lyophilized to obtain the peptide.

Step 7: The compound (˜6 mg) was dissolved in 8 mL of MeOH:H₂O (0.001 M HCl) (1:1). The solution was hydrogenated in the presence of 1-2 mg of PtO₂ (Adams' catalyst) for 10-12 hours. Catalyst was filtered and the solution was evaporated to dryness. The residue was dissolved in 1-2 mL of water and evaporated and the process was repeated two more times. The residue was dissolved in water and lyophilized to obtained the product.

### EXAMPLE 3

Somatostatins

(AA)ₐ is Phe, Tyr, an isomer of Tyr, polyhydroxylated Phe or aromatic amino acids, wherein the amino acid can have an L- or D-configuration;

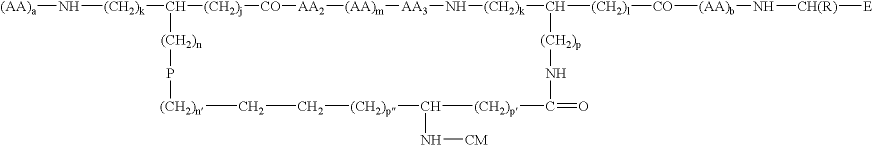

k is 1, 2or 3;

l is 1, 2 or 3;

AA₂ is Phe, Tyr, an isomer of Tyr, polyhydroxylated Phe or aromatic amino acids, wherein the amino acid can have an L- or D-configuration;

(AA)ₘ is a dipeptide sequence consisting of DTrp-Lys, DTrp-Om, DTrp-Dab, DTrp-4-piperidinylglycine, DTrp-4-piperidinylalanine, DTrp-4-aminomethylcyclohexy-lalanine, DTrp-4-aminomethylcyclohexylglycine, DTrp-4-aminocyclohexylalanine, DTrp-4-aminocyclohexylglycine and DTrp can be substituted by L-Trp;

AA₃ is any amino acid;

(AA)_{b} is none, serine or threonine;

R is hydrogen or C₁-C₅ linear or branched chain alkyl groups bearing —OH at any location;

E is COOH, CH₂—OH, CONH₂, COOR₄ or CONHOH wherein R₄ is hydrogen or C₁-C₅ linear or branched chain alkyl groups;

n is 1, 2 or 3;

P is none, O or S;

n′ is 1-7;

p is 1-6;

p′ is 1-6;

p″ is 1-6;

CM is a dye, a therapeutic agent, or a chelating moiety or metal binding site wherein the chelating moiety is labeled with a metal isotope selected from ^{99m}Tc, ²⁰³Pb, ⁶⁷Ga, ¹¹¹In, ⁹⁷Ru, ⁶²Cu, ⁶⁴Cu, ¹⁸⁶Re, ¹⁸⁸Re, ⁹⁰Y, ¹²¹Sn, ¹⁶¹Tb, ¹⁵³Sm, 166Ho, ¹⁰⁵Rh, ¹⁷⁷Lu or a radioactive halogen isotope on the understanding that

i) if the label is a metal isotope, CM represents a chelating group suitable for the metal and

ii) if the label is a radioactive halogen isotope, the halogen is attached to an aromatic ring,

wherein the CM is attached directly or through a spacing group to the peptide, said CM being attached to the amine through an amnide or urea bond or by any other modification which allows attachment of a chelate and which modifications are known to those of skill in the art,

wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA) or a compound with a general formula

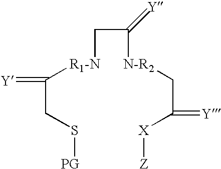

wherein

PG is a sulfur protecting group selected from alkanoyl, arylcarbonyl, arylaLkanoyl, acetamidomethyl, tetrahydropyranyl and tetrahydrofuranyl,

Y′, Y″, and Y′″ are hydrogen or oxygen with the proviso that at least one of them is an O,

R₁ and R₂ are hydrogen or alkyl (C₁-C₃),

X═NH or S with the proviso that Y′″ is hydrogen when X is S,

Z is PG if X is S, and

Z is hydroxyalkyl, aminoalkyl or carboxyalkyl.

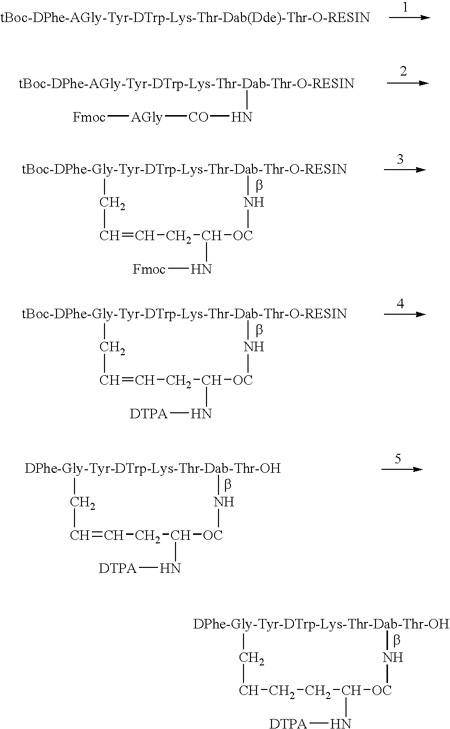

Step 1: The protected peptide was assembled in an automated synthesizer according to the Fmoc-strategy. The resin bound peptide was shaken with 2% hydrazine (2 mL per 50 mg resin) for 30 minutes to remove the Dde protecting group, followed by reaction with Fmoc-L-allylglycine activated ester (4 fold excess) to give the product.

Step, 2: 50 mg of the resin (25 μmole peptide) was suspended in 5 mL of methylene chloride containing 20 mg of Grubbs' catalyst. The mixture was heated at 40° C. for 10-15 hours. At the end of the reaction, the resin was removed by filtration and washed with methylene chloride and THF.

Step 3: The resin was shaken with 1:1 piperidine:DMF (1 mL per 50 mg resin) for 1 hour. After the resin was filtered it was washed with THF and dried. A solution of tri-t-butyl-DTPA anhydride (56 mg; 0.1 mmol) in 200 μL of DMF was activated with 0.5 mL of HOBt-HBTU (200 mM) solution for 1 hour and added to 140 mg (50 μmol of the peptide) of the above resin. The suspension was shaken for overnight and filtered. The resin was washed with DMF and 10 mL of THF.

Step 4: The resin was deprotected using 250 μL of TFA:phenol:thioanisole:water (85:5:5:5) overnight. The crude peptide was precipitated using 10 mL of MeOtBu. After centrifugation, the mixture was washed with 4×10 mL of MeOtBu. The mixture was taken up in 2 mL of 2:3 acetonitrile:water, shaken in a vortex mixer and the resin was removed by filtration. The filtrate was lyophilized to obtain the peptide.

Step 5: The compound (˜5 mg) was dissolved in 10 mL of MeOH:H₂O (0.001M HCl) (1:1). The solution was hydrogenated in the presence of 1-2 mg of PtO₂ (Adams' catalyst) for 10-12 hours. Catalyst was filtered and the solution was evaporated to dryness. The residue was dissolved in 1-2 mL of water and evaporated and the process was repeated two more times. The residue was dissolved in water and lyophilized to obtained the product.

### EXAMPLE 4

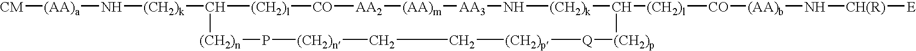

AA, AA₂, AA₃=natural and unnatural amino acids; this includes α-, β- and γ-aminoacids and L- and D-aminoacids;

a, b=0-10;

k, l=0-5;

m=0-20;

n, n′=1-10;

P, Q is none, O, S, COO, NH—CO, NR, N—CH(═NH)—NH₂, NH—CO—NH, NH—COO;

R is hydrogen or C₁-C₅ linear or branched chain alkyl groups bearing —OH at any location;

p, p′=0-10;

E is a group of formula COOR₄, CH₂OR₅, CON(R₆)OH, CON(R₇)(R₈) wherein

R₄ is hydrogen or C₁-C₅ linear or branched chain alkyl groups,

R₅ is hydrogen or physiologically acceptable, physiologically hydrolyzable ester,

R₆ is hydrogen or C₁-C₅ linear or branched chain alkyl groups,

R₇, R₈ is hydrogen or Cl-C₅ linear or branched chain alkyl groups or taken together form a cyclic alkyl group C₃-C₁₀;

CM is a dye, a therapeutic agent, or a chelating moiety or metal binding site wherein the chelating moiety is labeled with a metal isotope selected from ^{99m}Tc, ²⁰³Pb, 67Ga, ¹¹¹In, ⁹⁷Ru, ⁶²Cu, ⁶⁴Cu, ¹⁸⁶Re, ¹⁸⁸Re, ⁹⁰Y, ¹²¹Sn, ¹⁶¹Tb, ¹⁵³Sm, ¹⁶⁶Ho, ¹⁰⁵Rh, ¹⁷⁷Lu or a radioactive halogen isotope on the understanding that

i) if the label is a metal isotope, CM represents a chelating group suitable for the metal and

ii) if the label is a radioactive halogen isotope, the halogen is attached to an aromatic ring,

wherein the CM is attached directly or through a spacing group to the peptide, said CM being attached to the amine through an amide or urea bond or by any other modification which allows attachment of a chelate and which modifications are known to those of skill in the art,

wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA) or a compound with a general formula

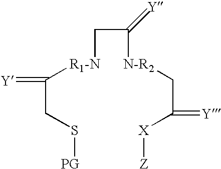

wherein

PG is a sulfur protecting group selected from alkanoyl, arylcarbonyl, arylalkanoyl, acetamidomethyl, tetrahydropyranyl and tetrahydrofuranyl,

Y′, Y″, and Y′″ are hydrogen or oxygen with the proviso that at least one of them is an O,

R₁ and R₂ are hydrogen or alkyl (C₁-C₃),

X═NH or S with the proviso that Y′″ is hydrogen when X is S,

Z is PG if X is S, and

Z is hydroxyalkyl, aminoalkyl or carboxyalkyl.

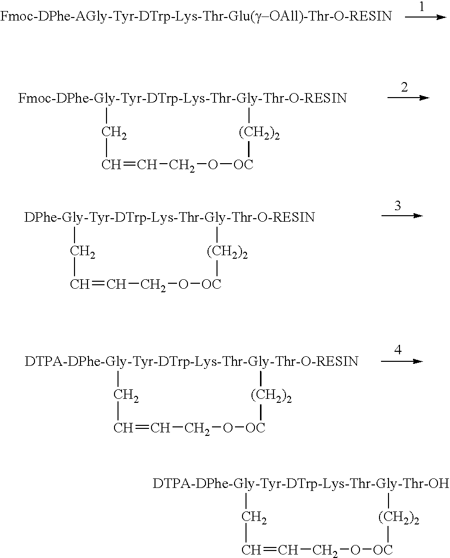

Step 1: 500 mg of the resin (90 μmole peptide) was suspended in 22 mL of methylene chloride containing 90 mg of Grubbs' catalyst. The mixture was heated at 40° C. for 10 hours. At the end of the reaction, the resin was removed by filtration and washed with methylene chloride and THF.

Step 2: The resin containing the cyclic product was treated with 5 mL of 1:1 piperidine:DMF for 30 minutes and filtered. The resin was washed with DMF and 10 mL of anhydrous THF and dried.

Step 3: A solution of tri-t-butyl-DTPA anhydride (112 mg; 0.2 mmol) in 200 μL of DMF was activated with 1 mL of HOBt-HBTU (200 mM) solution for 1 hour and added to 277 mg (50 μmol of the peptide) of the above resin. The suspension was shaken for overnight and filtered. The resin was washed with DMF and 10 mL of THF.

Step 4: The resin (9 μmole; 50 mg; 0.18 mmol/g) was suspended in a solution of 1 mL of DMF containing 30 mg (180 μmole) of p-fluorobenzenesulfonylhydrazide and heated at 75° C. for 6 hours. The resin was filtered, washed successively with 5 mL each of DMF and THF and dried. The deprotections were accomplished by using 250 μL of TFA:phenol:thioanisole:water (85:5:5:5) overnight. The crude peptide was precipitated using 10 mL of MeOtBu. After centrifugation, the mixture was washed with 4×10 mL of MeOtBu. The mixture was taken up in 2 mL of 2:3 acetonitrile:water, shaken in a vortex mixer and the resin was removed by filtration. The filtrate was lyophilized to obtain the peptide.

In a similar fashion, the following reactions were performed illustrating the use of these reactions to form a macrocycle containing two ester bonds (i.e., both P and Q are esters). Only some of the reaction steps are shown and are described below. Addition of a dye, therapeutic agent or chelating moiety can be performed as described above. This illustrates the generality of the reactions.

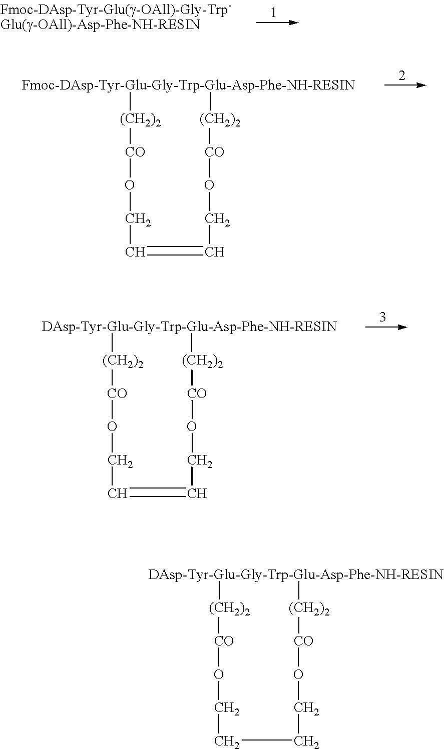

Step 1: 500 mg of the resin (90 μmole peptide) was suspended in 22 mL of methylene chloride containing 90 mg of Grubbs' catalyst. The mixture was heated at 40° C. for 10 hours. At the end of the reaction, the resin was removed by filtration and washed with methylene chloride and THF.

Step 2: The resin containing the cyclic product was treated with 5 mL of 1:1 piperidine:DMF for 30 minutes and filtered. The resin was washed with DMF and 10 mL of anhydrous THF and dried.

Step 3: The resin (9 μmole; 50 mg; 0.18 mmol/g) was suspended in a solution of 1 mL of DMF containing 30 mg (180 μmole) of p-fluorobenzenesulfonylhydrazide and heated at 75° C. for 6 hours. The resin was filtered, washed successively with 5 mL each of DMF and THF and dried. The deprotections were accomplished by using 250 μL of TFA:phenol:thioanisole:water (85:5:5:5) overnight. The crude peptide was precipitated using 10 mL of MeOtBu. After centrifugation, the mixture was washed with 4×10 mL of MeOtBu. The mixture was taken up in 2 mL of 2:3 acetonitrile:water, shaken in a vortex mixer and the resin was removed by filtration. The filtrate was lyophilized to obtain the peptide.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

What is claimed is:

1. A peptide of formula 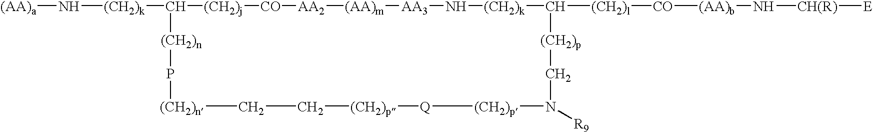
wherein
AA, AA₂, AA₃ are natural or unnatural amino acids comprising α-, β- and γ-aminoacids and L- and D-aminoacids;
a, b=0-10;
k, l=0-5;
m=0-20;
n, n′=1-10;
P is none, O, S, COO, NH—CO, NR, N—CH(═NH)—NH₂, NH—CO—NH, NH—COO;
R is hydrogen or C₁-C₅ linear or branched chain alkyl groups bearing —OH at any location;
p, p′, p″=0-10;
Q is none, O, S, COO, NH—CO, NR, N—CH(═NH)—NH₂;
E is a group of formula COOR₄, CH₂OR₅, CON(R₆)OH or CON(R₇)(R₈) wherein
R₄ is hydrogen or C₁-C₅ linear or branched chain alkyl groups,
R₅ is hydrogen or physiologically acceptable, physiologically hydrolyzable ester,
R₆ is hydrogen or C₁-C₅ linear or branched chain alkyl groups,
R₇, R₈ is hydrogen or C₁-C₅ linear or branched chain alkyl groups or taken together form a cyclic alkyl group C₃-C₁₀; and
R₉ is H, a dye, a therapeutic agent, a chelating moiety or a metal binding site.

2. The peptide of claim 1 wherein said chelating moiety or metal binding site is CM and CM is labeled with a metal isotope selected from ^{99m}Tc, ²⁰³Pb, ⁶⁷Ga, 111In, ⁹⁷Ru, ⁶²Cu, ⁶⁴Cu, ¹⁸⁶Re, ¹⁸⁸Re ⁹⁰Y, ¹²¹Sn, ¹⁶¹Tb, ¹⁵³Sm, ¹⁶⁶Ho, ¹⁰⁵Rh, ¹⁷⁷Lu or a radioactive halogen isotope on the understanding that
i) if the label is a metal isotope, CM represents a chelating group suitable for the metal and
ii) if the label is a radioactive halogen isotope, the halogen is attached to an aromatic ring,
wherein the CM is attached directly or through a spacing group to the peptide, said CM being attached to the amine through an amide or urea bond or by any other modification which allows attachment of a chelate and which modifications are known to those of skill in the art,
wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA) or a compound with a general formula 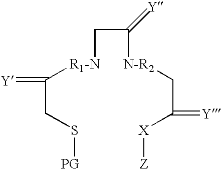
wherein
PG is a sulfur protecting group selected from alkanoyl, arylcarbonyl, arylalkanoyl, acetamidomethyl, tetrahydropyranyl and tetrahydrofuranyl,
Y′, Y″, and Y′″ are hydrogen or oxygen with the proviso that at least one of them is an O,
R₁ and R₂ are hydrogen or alkyl (C₁-C₃),
X═NH or S with the proviso that Y′″ is hydrogen when X is S,
Z is PG if X is S, and
Z is hydroxyalkyl, aminoalkyl or carboxyalkyl.

3. The peptide of claim 1 wherein said dye is selected from the group consisting of fluorescein, fluorescein isothiocyanate, naphthofluorescein, rhodarnine derivatives, Texas Red, hydroxycoumarin, indocyanine green, bis-propanoic acid cyanine, acridines, thiazines, phenazines, porphyrins and naphthalimide.

4. The peptide of claim 1 wherein said therapeutic agent is selected from the group consisting of tamoxifen, adriamycin, phillotoxins, taxol, taxol analogs, bleomycin, doxorubicin, etoposide, methotrexate, vinblastine, vinblastine analogs, dicarbazine and actinomycin D.

5. The peptide of claim 1 wherein said peptide is a derivative of: somatostatin, gastrin, gastrin releasing peptide, bombesin, a bombesin antagonist, a gastrin releasing peptide, an adhesion peptide, cholecystokinin, a neurotensin, neuropeptide Y, a vasoactive intestinal peptide, thyroid stimulating hormone, angiotensin, pancreatic adenylate cyclase activating peptide or substance P.

6. A peptide of formula 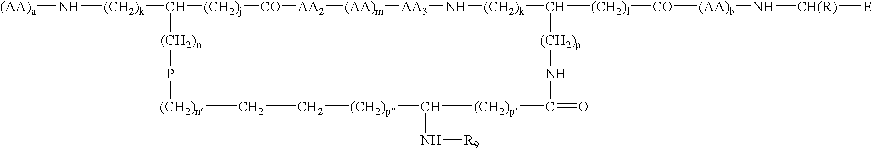
wherein
AA, AA₂, AA₃ are natural or unnatural amino acids comprising α-, β- or γ-aminoacids, and L- or D-aminoacids;
a, b=0-10;
k, l=0-5;
m=0-20;
n, n′=1-10;
P is none, O, S, COO, NH—CO, NR, N—CH(═NH)—NH₂, NH—CO—NH, NH—COO;
R is hydrogen or C₁-C₅ linear or branched chain alkyl groups bearing —OH at any location;
p, p′, p″=0-10;
E is a group of formula COOR₄, CH₂OR₅, CON(R₆)OH or CON(R₇)(R₈) wherein
R₄ is hydrogen or C₁-C₅ linear or branched chain alkyl groups,
R₅ is hydrogen or physiologically acceptable, physiologically hydrolyzable ester,
R₆ is hydrogen or C₁-C₅ linear or branched chain alkyl groups,
R₇, R₈ is hydrogen or C₁-C₅ linear or branched chain alkyl groups or taken together form a cyclic alkyl group C₃-C₁₀; and
R₉ is H, a dye, a therapeutic agent, a chelating moiety or a metal binding site.

7. The peptide of claim 6 wherein said chelating moiety or metal binding site is CM and CM is labeled with a metal isotope selected from ^{99m}Tc, ²⁰³Pb, ⁶⁷Ga, ¹¹¹In, ⁹⁷Ru, ⁶²Cu, ⁶⁴Cu, ¹⁸⁶Re, ¹⁸⁸Re, ⁹⁰Y, ¹²¹Sn, ¹⁶¹Tb, ¹⁵³Sm, ¹⁶⁶Ho, ¹⁰⁵Rh, ¹⁷⁷Lu or a radioactive halogen isotope on the understanding that
i) if the label is a metal isotope, CM represents a chelating group suitable for the metal and
ii) if the label is a radioactive halogen isotope, the halogen is attached to an aromatic ring,
wherein the CM is attached directly or through a spacing group to the peptide, said CM being attached to the amine through an amide or urea bond or by any other modification which allows attachment of a chelate and which modifications are known to those of skill in the art,
wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA) or a compound with a general formula 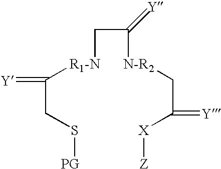
wherein
PG is a sulfur protecting group selected from alkanoyl, arylcarbonyl, arylalkanoyl, acetamidomethyl, tetrahydropyranyl and tetrahydrofuranyl,
Y′, Y″, and Y′″ are hydrogen or oxygen with the proviso that at least one of them is an O,
R₁ and R₂ are hydrogen or alkyl (C₁-C₃),
X═NH or S with the proviso that Y′″ is hydrogen when X is S,
Z is PG if X is S, and
Z is hydroxyalkyl, aminoalkyl or carboxyalkyl.

8. The peptide of claim 6 wherein said dye is selected from the group consisting of fluorescein, fluorescein isothiocyanate, naphthofluorescein, rhodamine derivatives, Texas Red, hydroxycoumarin, indocyanine green, bis-propanoic acid cyanine, acridines, thiazines, phenazines, porphyrins and naphthalimide.

9. The peptide of claim 6 wherein said therapeutic agent is selected from the group consisting of tamoxifen, adriamycin, phillotoxins, taxol, taxol analogs, bleomycin, doxorubicin, etoposide, methotrexate, vinblastine, vinblastine analogs, dicarbazine and actinomycin D.

10. The peptide of claim 6 wherein said peptide is a derivative of: somatostatin, gastrin, gastrin releasing peptide, bombesin, a bombesin antagonist, a gastrin releasing peptide, an adhesion peptide, cholecystokinin, a neurotensin, neuropeptide. Y, a vasoactive intestinal peptide, thyroid stimulating hormone, angiotensin, pancreatic adenylate cyclase activating peptide or substance P.

11. A peptide of formula 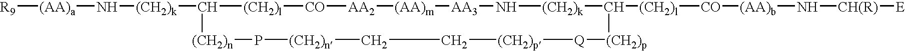
wherein
AA, AA₂, AA₃ are natural and unnatural amino acids comprising α-, β- or γ-aminoacids and L- and D-aminoacids;
a, b=0-10;
k, l=0-5;
m=0-20;
n, n′=1-10;
P, Q is none, O, S, COO, NH—CO, NR, N—CH(═NH)—NH₂, NH—CO—NH, NH—COO;
R is hydrogen or C₁-C₅ linear or branched chain alkyl groups bearing —OH at any location;
p, p′=0-10;
E is a group of formula COOR₄, CH₂OR₅, CON(R₆)OH or CON(R₇)(R₈) wherein
R₄ is hydrogen or C₁-C₅ linear or branched chain alkyl groups,
R₅ is hydrogen or physiologically acceptable, physiologically hydrolyzable ester,
R₆ is hydrogen or C₁-C₅ linear or branched chain alkyl groups,
R_{7,} R₈ is hydrogen or C₁-C₅ linear or branched chain alkyl groups or taken together form a cyclic alkyl group C₃-C₁₀; and
R₉ is H, a dye, a therapeutic agent, a chelating moiety or a metal binding site.

12. The peptide of claim 11 wherein said chelating moiety or metal binding site is CM wherein CM is labeled with a metal isotope selected from ^{99m}Tc, ²⁰³Pb, ⁶⁷Ga, ¹¹¹In, ⁹⁷Ru, ⁶²Cu, ⁶⁴Cu, ¹⁸⁶Re, ¹⁸⁸Re, ⁹⁰Y, ¹²¹Sn, ¹⁶¹Tb, ¹⁵³Sm, ¹⁶⁶Ho, ¹⁰⁵Rh, ¹⁷⁷Lu or a radioactive halogen isotope on the understanding that
i) if the label is a metal isotope, CM represents a chelating group suitable for the metal and
ii) if the label is a radioactive halogen isotope, the halogen is attached to an aromatic ring,
wherein the CM is attached directly or through a spacing group to the peptide, said CM being attached to the amine through an amide or urea bond or by any other modification which allows attachment of a chelate and which modifications are known to those of skill in the art,
wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA) or a compound with a general formula 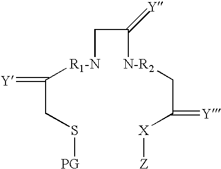
wherein
PG is a sulfur protecting group selected from alkanoyl, arylcarbonyl, arylalkanoyl, acetamidomethyl, tetrahydropyranyl and tetrahydrofuranyl,
Y′, Y″, and Y′″ are hydrogen or oxygen with the proviso that at least one of them is an O,
R₁ and R₂ are hydrogen or alkyl (C₁-C₃),
X═NH or S with the proviso that Y′″ is hydrogen when X is S,
Z is PG if X is S, and
Z is hydroxyalkyl, aminoalkyl or carboxyalkyl.

13. The peptide of claim 11 wherein said dye is selected from the group consisting of fluorescein, fluorescein isothiocyanate, naphthofluorescein, rhodamine derivatives, Texas Red, hydroxycoumarin, indocyanine green, bis-propanoic acid cyanine, acridines, thiazines, phenazines, porphyrins and naphthalimide.

14. The peptide of claim 11 wherein said therapeutic agent is selected from the group consisting of tamoxifen, adriamycin, phillotoxins, taxol, taxol analogs, bleomycin, doxorubicin, etoposide, methotrexate, vinblastine, vinblastine analogs, dicarbazine and actinomycin D.

15. The peptide of claim 11 wherein said peptide is a derivative of: somatostatin, gastrin, gastrin releasing peptide, bombesin, a bombesin antagonist, a gastrin releasing peptide, an adhesion peptide, cholecystokinin, a neurotensin, neuropeptide Y, a vasoactive intestinal peptide, thyroid stimulating hormone, angiotensin, pancreatic adenylate cyclase activating peptide or substance P.

16. A method for labeling a peptide with a dye, a therapeutic agent, a chelating moiety or a metal binding site to create a labeled peptide, said method comprising:
a) synthesizing a macrocyclic ring on said peptide wherein said ring comprises a functional group to which said dye, therapeutic agent, chelating moiety or metal binding site can be attached; and
b) attaching said dye, therapeutic agent, chelating moiety or metal binding site to said peptide.

17. The method of claim 16 wherein step (a) is performed using a metathesis reaction.

18. The method of claim 17 wherein Grubbs' catalyst is used to catalyze the reaction.

19. The method of claim 16 wherein said labeled peptide is selected from the group consisting of: 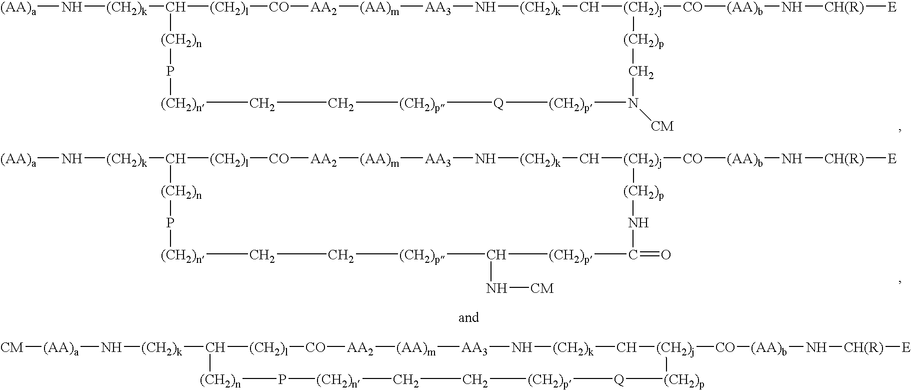
wherein
AA, AA₂, AA₃ are natural and unnatural amino acids comprising α-, β- or γ-aminoacids and L- and D-aminoacids;
a, b=0-10;
k, l=0-5;
m=0-20;
n, n′=1-10;
P, Q is none, O, S, COO, NH—CO, NR, N—CH(═NH)—NH₂, NH—CO—NH, NH—COO;
R is hydrogen or C₁-C₅ linear or branched chain alkyl groups bearing —OH at any location;
p, p′, p″=0-10;
E is a group of formula COOR₄, CH₂OR₅, CON(R₆)OH or CON(R₇)(R₈) wherein
R₄ is hydrogen or C₁-C₅ linear or branched chain alkyl groups,
R₅ is hydrogen or physiologically acceptable, physiologically hydrolyzable ester,
R₆ is hydrogen or C₁-C₅ linear or branched chain alkyl groups,
R₇, R₈ is hydrogen or C₁-C₅ linear or branched chain alkyl groups or taken together form a cyclic alkyl group C₃-C₁₀; and
CM is a chelating moiety or metal binding site wherein the chelating moiety is labeled with a metal isotope selected from ^{99m}Tc, ²⁰³Pb, ⁶⁷Ga, ¹¹¹In, ⁹⁷Ru, ⁶²Cu, ⁶⁴Cu, ¹⁸⁶Re, 188Re, ⁹⁰Y, ¹²¹Sn, ¹⁶¹Tb, ¹⁵³Sm, ¹⁶⁶Ho, ¹⁰⁵Rh, ¹⁷⁷Lu or a radioactive halogen isotope on the understanding that
i) if the label is a metal isotope, CM represents a chelating group suitable for the metal and
ii) if the label is a radioactive halogen isotope, the halogen is attached to an aromatic ring,
wherein the CM is attached directly or through a spacing group to the peptide, said CM being attached to the amine through an amide or urea bond or by any other modification which allows-attachment of a chelate and which modifications are known to those of skill in the art,
wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (IOTA), 1,4,7-triazacyclononane-N,N′,N″-triacetic acid ROTA), 1,4,8,11-tetraazacyclotetradecane-N,N′,N″,N′″-tetraacetic acid (TETA) or a compound with a general formula 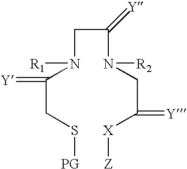
wherein
PG is a sulfur protecting group selected from alkanoyl, arylcarbonyl, arylalkanoyl, acetamidomethyl, tetrahydropyranyl and tetrahydrofuranyl,
Y′, Y″, and Y′″ are hydrogen or oxygen with the proviso that at least one of them is an O,
R₁ and R₂ are hydrogen or alkyl (C₁-C₃),
X═NH or S with the proviso that Y′″ is hydrogen when X is S,
Z is PG if X is S, and
Z is hydroxyalkyl, aminoalkyl or carboxyalkyl.

20. The method of claim 16 wherein said peptide is a derivative of: somatostatin, gastrin, gastrin releasing peptide, bombesin, a bombesin antagonist, a gastrin releasing peptide, an adhesion peptide, cholecystokinin, a neurotensin, neuropeptide Y, a vasoactive intestinal peptide, thyroid stimulating hormone, angiotensin, pancreatic adenylate cyclase activating-peptide or substance P.

21. The method of claim 16 wherein said dye is selected from fluorescein, fluorescein isothiocyanate, naphthofluorescein, rhodamine derivatives, Texas Red, hydroxycoumarin, indocyanine green, bis-propanoic acid cyanine, acridines, thiazines, phenazines, porphyrins and naphthalimide.

22. The method of claim 16 wherein said therapeutic agent is selected from tamoxifen, adriamycin, phillotoxins, taxol, taxol analogs, bleomycin, doxorubicin, etoposide, methotrexate, vinblastine, vinblastine analogs, dicarbazine and actinomycin D.

23. The method of claim 16 wherein said chelating moiety or metal binding agent is CM and CM is labeled with a metal isotope selected from ^{99m}Tc, ²⁰³Pb, ⁶⁷Ga, ¹¹¹In, ⁹⁷Ru, ⁶²Cu, ⁶⁴Cu, ¹⁸⁶Re, ¹⁸⁸Re, ⁹⁰Y, ¹²¹Sn, ¹⁶¹Tb, ¹⁵³Sm, ¹⁶⁶Ho, ¹⁰⁵Rh, ¹⁷⁷Lu or a radioactive halogen isotope on the understanding that
i) if the label is a metal isotope, CM represents a chelating group suitable for the metal and
ii) if the label is a radioactive halogen isotope, the halogen is attached to an aromatic ring,
wherein the CM is attached directly or through a spacing group to the peptide, said CM being attached to the amine through an amide or urea bond or by any other modification which allows attachment of a chelate and which modifications are known to those of skill in the art,
wherein the chelating group is preferably derived from ethylene diamine tetraacetic acid (EDTA), diethylene triamine pentaacetic acid (DTPA), cyclohexyl 1,2-diamine tetraacetic acid (CDTA), ethyleneglycol-O,O′-bis(2-aminoethyl)-N,N,N′,N′-diacetic acid (HBED), triethylene tetraamine hexaacetic acid (TTHA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA) or a compound with a general formula 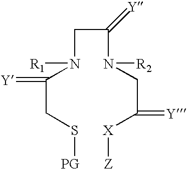
wherein
PG is a sulfur protecting group selected from alkanoyl, arylcarbonyl, arylalkanoyl, acetamidomethyl, tetrahydropyranyl and tetrahydrofuranyl,
Y′, Y″, and Y′″ are hydrogen or oxygen with the proviso that at least one of them is an O,
R₁ and R₂ are hydrogen or alkyl (C₁-C₃),
X═NH or S with the proviso that Y′″ is hydrogen when X is S,
Z is PG if X is S, and
Z is hydroxyalkyl, aminoalkyl or carboxyalkyl.

24. A pharmaceutical formulation comprising a peptide of claim 1, claim 6 or claim 11.

25. A method of therapeutically treating an animal, including a person, comprising administering a therapeutic amount of a peptide of claim 1, claim 6 or claim 11 to said animal.

26. A method of diagnosing an animal, including a person, comprising administering a peptide of claim 1, claim 6 or claim 11 to said animal.
